(19) United States
(12) Patent Application Publication
(10) Pub. No.: US 2024/0390177 A1
(43) Pub. Date: Nov. 28, 2024

(54) MALE URINE COLLECTION DEVICE USING WICKING MATERIAL

(71) Applicant: PUREWICK CORPORATION, Covington, GA (US)

(72) Inventor: Camille Rose Newton, Bonsall, CA (US)

(21) Appl. No.: 18/680,411

(22) Filed: May 31, 2024

Related U.S. Application Data
(63) Continuation of application No. 15/221,106, filed on Jul. 27, 2016, now Pat. No. 10,376,406.
(63) Continuation of application No. 16/449,039, filed on Jun. 21, 2019, now Pat. No. 12,029,678.

(51) Int. Cl. A61F 5/453; A01K 23/00; A61B 10/00; A61F 5/44; A61G 9/00

(52) U.S. Cl. CPC A61F 5/453; A61F 5/4404; A01K 23/00; A61B 10/007; A61G 9/006

(57) ABSTRACT

A urine collection device has a chamber assembly, which includes a layer of wicking material and porous material. The porous material is configured to form a chamber in which urine can be collected for transport; and the wicking material is disposed about the porous material. The chamber has a port for receiving a tube so that urine collected within the chamber can be transported from the chamber by being drawn from the chamber when a partial vacuum is applied within the chamber via the received tube. The chamber assembly is so dimensioned and configured that opposing portions of the assembly are sufficient adjacent as to define an opening through which the head a penis can be inserted. A layer of impermeable material is so attached to the chamber assembly as to cover one side of the opening and thereby provide a receptable for receiving the head of an inserted penis.

### BACKGROUND OF THE INVENTION

The invention generally pertains to using wicking material to collect urine for transport and is particularly directed to a device that can be used to so collect urine from the penis of a person or an animal in such a manner that the urine can be readily transported from the device as the urine is being collected.

A container for collecting urine and transporting the collected urine voided from a person's body is described in U.S. Pat. No. 8,287,508 to Robert A. Sanchez. The container described in said patent is made of plastic or some other material and defines a chamber for collecting urine. The container is closed, except for having an array of openings through which urine can be drawn into the chamber for collection and at least one port through which urine can be drawn away from the chamber by a transport tube inserted into the chamber. The exterior of the container is configured for enabling a moisture-wicking article to be secured over the array of openings and for enabling the secured moisture-wicking article to be disposed in contact with the region of a female body surrounding the urethral opening. A vacuum pump is attached to the transport tube in order to create a partial vacuum in the chamber in order to draw urine into the chamber for collection of the urine and in order to draw the collected urine away from the chamber.

### SUMMARY OF THE INVENTION

The invention provides a device that can be used to so collect urine flowing from the penis of a person or an animal in such a manner that the urine can be readily transported from the device as the urine is being collected, the device comprising: a chamber assembly in which wicking material is disposed about porous material that is configured to form a chamber in which urine can be collected for transport, with the chamber having a port for receiving a tube so that urine collected within the chamber can be transported from the chamber by being drawn from the chamber when a partial vacuum is applied within the chamber via a said received tube, and with the assembly being dimensioned so and configured that opposing portions of the assembly are sufficiently adjacent as to define an opening through which the head of a penis can be inserted; and a layer of impermeable material so attached to the chamber assembly as to cover one side of the opening and thereby provide a receptacle for receiving the head of a said inserted penis, from which receptacle urine flowing from said penis can be drawn through the wicking material and the porous material into the chamber when a said partial vacuum is applied within the chamber via said tube. The chamber includes spaces within the porous material and/or spaces between portions of the configured porous material.

The invention is particularly useful for persons or animals during various circumstances. These circumstances include a condition such as incontinence or a disability that limits or impairs mobility. These circumstances also include restricted travel conditions, such as sometimes experienced by pilots, drivers, workers in hazardous areas, etc. These circumstances further include collection of urine for monitoring purposes or clinical testing.

Additional features of the invention are described with reference to the detailed description.

### BRIEF DESCRIPTION OF THE DRAWING

FIG. 1 is a top view of an exemplary embodiment of a urine collection device according to the invention.

FIG. 2 is a (non-cross-hatched) sectional view taken along line 2-2 in FIG. 1 showing various components included in urine collection device shown in FIG. 1.

### DETAILED DESCRIPTION

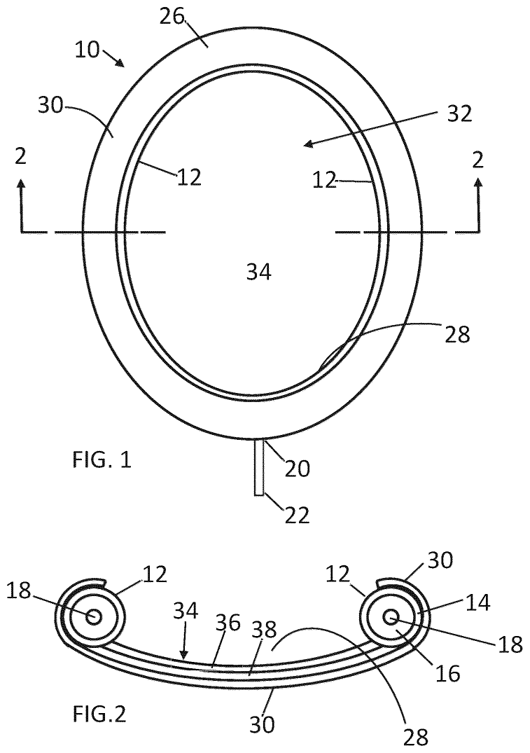

Referring to FIGS. 1 and 2, an exemplary embodiment of a unne collection device according to the invention includes a chamber assembly 12. The chamber assembly 12 includes a thin layer of wicking material 14 and a porous material 16. The porous material 16 is configured to form a continuous ring-like chamber 18 in which unne can be collected for transport; and the wicking material 14 is disposed about the porous material 16. The chamber 18 has a port 20 for receiving a tube 22 so that urine collected within the chamber 18 can be transported from the chamber 18 by being drawn from the chamber 18 when a partial vacuum is applied within the chamber 18 via the received tube 22. The received tube 22 extends past the port to within the chamber 18. The chamber assembly 12 is dimensioned and configured to define an opening 28 through which the head of a penis can be inserted.

In an alternative embodiment (not shown), the porous material is configured to form a discontinuous C-shaped chamber in which urine can be collected for transport, with opposing portions of the chamber assembly being sufficiently adjacent as to define an opening through which the head of a penis can be inserted.

A flexible sheet of impermeable material is so attached to the chamber assembly 12 as to cover one side of the opening 28 and thereby provide a receptacle 32 for receiving the head of an inserted penis. Urine flowing into the receptacle 32 from the penis can be drawn through the wicking material 14 and the porous material 16 into the chamber 18 when a partial vacuum is applied within the chamber 18 via the tube 22.

In the exemplary embodiment shown in FIG. 2, the sheet of impermeable material is so dimensioned in relation to the breadth of the opening 28 as to extend sufficiently away from the chamber assembly 12 as to provide adequate space in the receptacle to receive the head of the penis.

The layer of impermeable material 30 further covers at least the exterior sides of the chamber assembly 12; and in the exemplary embodiment shown in the Drawing, the layer of impermeable material 30 further covers a portion of the interior sides of the chamber assembly 12.

The layer of impermeable material 30 is so attached to the chamber assembly 12 by an adhesive material as to maintain the chamber assembly 12 in the configuration defining the opening 28, as shown in FIG. 1. In another embodiment (not shown), retainer clips or other fasteners attach the impermeable material to the chamber assembly.

The device 10 further includes a cushion 34, which is disposed in the receptacle 32 within the opening 28 for receiving the head of an inserted penis. The cushion 34 is so disposed over the layer of impermeable material 30 as to contact the wicking material 14 of the chamber assembly 12. The cushion 34 includes a layer of wicking material 36, such as medical gauze, disposed over a bed of porous material 38.

In the exemplary embodiment, the wicking material 14 is medical gauze. In other embodiments other wicking materials are used for the wicking material.

In the exemplary embodiment, the porous material 16 is provided as a web of a spun plastic material, such as nylon or polyester. In other embodiments, other materials are used as the porous material.

In the exemplary embodiment, the chamber 18 is formed by folding together opposite sides of a web of spun plastic material, whereby the chamber 18 does not necessarily have a closed cross-section as shown in FIG. 2.

In FIGS. 1 and 2, the relative dimensions of the various components are not shown to scale.

Different embodiments of a male urine collection device according to the invention are dimensioned and configured for use in both adult and pediatric applications, and for veterinary applications involving animals of different species and sizes.

When a man is lying on his back with the head of his penis disposed within the receptacle 32, urine flowing from the penis runs down the inner sides of the receptacle 32 between the layer of porous material and the sheet of impermeable material 30, through which the urine flows into the chamber 18 and thence to the outlet port 20. The urine collection device 10 can thus advantageously capture urine as it flows against gravity without having to attach a catheter to the penis.

The benefits specifically stated herein do not necessarily apply to every conceivable embodiment of the present invention. Further, such stated benefits of the present invention are only examples and should not be construed as the only benefits of the present invention.

While the above description contains much specificity, these specifics are not to be construed as limitations on the scope of the present invention, but rather as examples of the embodiments described herein. Other variations are possible and the scope of the present invention should be determined not by the embodiments described herein but rather by the claims and their legal equivalents.

1. (canceled)

2. A device to collect urine from a penis, the device comprising:
a porous material having a first side and a second side opposite the first side;
a wicking material positioned adjacent to substantially all of the first side of the porous material; and
a layer of impermeable material positioned at least proximate to at least a portion of the second side of the porous material;
wherein at least one of the porous material, the wicking material, or the layer of impermeable material defines an opening configured to receive a head of a penis, the wicking material positioned to directly receive urine from the head of the penis when the opening receives the head of the penis;
wherein the device defines a chamber including at least one substantially unoccupied gap, at least a portion of the chamber is spaced from and distinct from the port, at least a portion of the wicking material is spaced from at least a portion of the chamber by the porous material.

3. The device of claim 2, wherein the porous material exhibits a substantially uniform thickness.

4. The device of claim 2, wherein the porous material includes a spun plastic material.

5. The device of claim 2, wherein the porous material defines at least a portion of the chamber, at least a portion of the chamber is spaced from and distinct from a port configured to be attached to a tube.

6. The device of claim 2, wherein a portion of the porous material opposite the opening is a unitary, continuous layer.

7. The device of claim 2, wherein the wicking material is immediately adjacent to the opening.

8. The device of claim 2, wherein the wicking material includes a gauze.

9. The device of claim 2, wherein the wicking material includes a first portion and a second portion, a direction that the wicking material extends changes where the wicking material of the first portion and the second portion meet, the first portion and the second portion of the wicking material are positioned to directly receive urine from the head of the penis when the opening receive the head of the penis.

10. The device of claim 2, wherein the wicking material exhibits substantially uniform thickness.

11. The device of claim 2, wherein the layer of impermeable material contacts a portion of the porous material.

12. The device of claim 2, wherein the layer of impermeable material defines a portion configured to be attached to a tube.

13. The device of claim 2, wherein at least a portion of the chamber exhibits an annular shape with at least one of the porous material or the wicking material positioned within the annular shaped chamber.

14. A device to collect urine from a penis, the device comprising:
a porous material having a first side and a second side opposite the first side;
a wicking material positioned adjacent to substantially all of the first side of the porous material; and
a layer of impermeable material positioned at least proximate to at least a portion of the second side of the porous material; and
a port configured to be attached to a tube;
wherein a portion of the porous material, a portion of the wicking material, and a portion of the layer of impermeable material that are opposite the opening extend substantially parallel to each other;
wherein at least one of the porous material, the wicking material, or the layer of impermeable material defines an opening configured to receive a head of a penis, the wicking material positioned to directly receive urine from the head of the penis when the opening receives the head of the penis;
wherein the device defines a chamber between the layer of impermeable material and at least one of the wicking material or the porous material, the chamber including at least one substantially unoccupied gap, at least a portion of the chamber is spaced from and distinct from the port.

15. The device of claim 13, wherein at least a portion of the chamber is directly defined by the porous material.

16. The device of claim 2, wherein the porous material includes a spun plastic material.

17. The device of claim 2, wherein a portion of the porous material opposite the opening is a unitary, continuous layer.

18. The device of claim 2, wherein at least a portion of the chamber exhibits an annular shape with at least one of the porous material or the wicking material positioned within the annular shaped chamber.

19. A device to collect urine from a penis, the device comprising:
a porous material having a first side and a second side opposite the first side;
a wicking material positioned adjacent to substantially all of the first side of the porous material; and
a layer of impermeable material positioned at least proximate to at least a portion of the second side of the porous material; and
a port configured to be attached to a tube;
wherein at least one of the porous material, the wicking material, or the layer of impermeable material defines an opening configured to receive a head of a penis, the wicking material positioned to directly receive urine from the head of the penis when the opening receives the head of the penis;
wherein the device defines a chamber including at least one substantially unoccupied gap, at least a portion of the chamber is directly defined by the porous material, at least a portion of the chamber is spaced from and distinct from the port.

20. The device of claim 2, wherein a portion of the porous material opposite the opening is a unitary, continuous layer.

21. The device of claim 2, wherein the porous material includes a spun plastic material.
